(19) United States
(12) Patent Application Publication
(10) Pub. No.: US 2026/0354982 A1
(43) Pub. Date: Nov. 12, 2026

(54) DUAL MOBILITY HIP PROSTHESIS

(71) Applicant: David Kirwan, WIRLINGA (AU)

(72) Inventor: David Kirwan, WIRLINGA (AU)

(21) Appl. No.: 19/131,732

(22) PCT Filed: Dec. 5, 2023
(86) PCT No.: PCT/AU2023/051253

§ 371 Date: May 21, 2025

(30) Foreign Application Priority Data
Dec. 14, 2022 (AU) 2022903837

(51) Int. Cl. A61F 2/36; A61F 2/34

(52) U.S. Cl. CPC A61F 2/3609; A61F 2/34; A61F 2002/3443

(57) ABSTRACT

The present dual mobility bearing component has geometry featuring a high-sided and low-ended aperture which aims to reduce complications associated with conventional dual mobility prostheses through compartmentalisation of torque, shear stress, and movements between the inner and outer bearings, minimising cross-shear and wear. This geometry induces flexion and extension movements of the femoral component to occur primarily along a wider axis of the aperture, an adduction and abduction movement to predominate along a relatively narrower axis and being generally restricted to the outer bearing surface of the bearing component, induced by the impingement of the femoral component's neck against the high sides of the rim along the narrower axis.

### FIELD OF THE INVENTION

This invention relates generally to a type of bearing component for dual mobility hip prosthesis.

### BACKGROUND OF THE INVENTION

Total Hip Replacement is a highly successful procedure, which involves insertion of prosthetic components, into and for treatment of, a diseased hip.

Components include a stem of a femoral component and a socket of an acetabular component. These two sides of the joint articulate via bearing components. The bearings usually comprise a head on the femoral side and a liner on the acetabular side.

If a neck of the femoral component and the rim of the acetabular component make contact, cervico-acetabular prosthetic impingement is said to occur. Prosthetic impingement is very harmful because it can cause component wear, damage, instability and failure. These complications frequently result in extensive and further surgical intervention (revision surgery).

To avoid prosthetic impingement, hip replacements may be designed with a much higher head-to-neck diameter ratio as compared to that occurring in the native hip. Ideally, bone shape and soft tissue tension prevent an excess range of motion or limit motion before prosthetic impingement can occur. Yet, prosthetic impingement and other problems still occur.

Dual mobility prostheses involve the interposition of an extra and intermediate dual mobility bearing component. The effect of a dual mobility bearing component is to improve (i.e., increase) the effective head-to-neck diameter ratio and so further avoid prosthetic impingement. Routinely, dual mobility bearing components are made from High Density Polyethylene (HDPE).

Dual mobility prostheses were originally developed in France and were intended for use in elderly patients only. However, due to their success, the use of dual mobility prostheses has increased in recent years in many countries and in younger patients, to further improve results of total hip replacement.

The dual mobility bearing component circumscribes the femoral head by more than 180° in cross-section to thereby lock onto the head and so that the head can articulate within the dual mobility bearing component at an inner bearing. The dual mobility bearing component is then rested or passively reduced into the acetabular liner component, where it can also rotate at an outer bearing. Torque stresses occur at both the inner bearing and the outer bearing (whether either bearing is moving or not).

However, due to a difference in frictional torque (a consequence of the different radii), most movement occurs at the inner bearing. The outer bearing only moves passively, if the prosthetic femoral neck abuts the rim of the dual mobility bearing component (i.e., middle bearing). Also, the predominant movement occurring at the inner bearing is rotation.

These movements and torque stresses in multiple directions cause more frictional wear in HDPE than when these stresses are aligned. Such so-called “cross-shear” is especially harmful for HDPE, in terms of (frictional) wear. Poly wear is a well-recognised problem of conventional hip replacement involving dual mobility bearing components.

The present invention seeks to provide a way, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

While dual mobility prostheses have been successful in improving the head-to-neck diameter ratio, an incidence of some complications has arisen which are peculiar to the use of dual mobility prostheses. Also, the expanded use of dual mobility prostheses in younger and more active patients than was originally intended has resulted in an increased incidence of these complications.

There is provided herein a HDPE (or any other suitable material) dual mobility bearing component designed to reduce many of these complications. It is contemplated that the present dual mobility bearing component will be suitable for safe use in younger and more active patients, rather than be restricted for use in the elderly as was originally intended.

The present dual mobility bearing component has a distinct geometry as compared to conventional dual mobility bearing components, in that it has a high sided aperture, with a low end height. The end heights may be generally similar to those of a generic component, but the effect of the higher sides is to partially envelop the head more than a generic dual mobility bearing component does.

Specifically, rather than appearing like a conventional ¾ spherical cap with a circular aperture, the present dual mobility bearing component has a “football stadium-like” or a “clamshell-like” appearance and shape, with high side walls and low end walls.

As such, it is envisaged that torque, shear stress and movements at the inner bearing and outer bearing will be substantially compartmentalised, such that cross-shear and cross-shear induced wear are substantially reduced.

Specifically, the present geometry induces flexion and extension torque moments and movements to predominantly occur at the inner bearing (along with rotation) whereas abduction and adduction movements will be induced to predominantly occur at the outer bearing, due to impingement of the prosthetic neck along the side-line or the high wall of the dual mobility bearing component.

In this way, the two components of cross-shear are stratified or separated to occur at the inner bearing and outer bearing respectively.

The rim may have inner flat contact faces such that engagement at the middle bearing occurs between two parallel edges and surfaces of the neck and the dual mobility bearing component respectively.

Accordingly, when the prosthetic neck drives the outer bearing to move, contact stresses are mitigated at the middle bearing and torque load at the inner bearing is significantly reduced.

Furthermore, the shape of the present dual mobility bearing component results in an aperture which is elliptical rather than round in plan view. As such, the non-circular in plan cross section and non-coplanar inner edge of the rim of the present dual mobility bearing component spreads and reduces insertion forces required to stretch the dual mobility bearing component onto the head during insertion.

This arrangement also increases the extraction forces required to remove the head from the dual mobility bearing component. Stated differently, the present dual mobility bearing component can be more firmly secured to the head to prevent dissociation, which by definition is a failure that requires revision surgery. In addition, the displacement necessary through which the head must travel for dissociation to occur is increased by the present design as compared to that of a generic dual mobility bearing component.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective view of a dual mobility bearing component in accordance with an embodiment;

FIG. 2 shows the dual mobility bearing component in accordance with a further embodiment;

FIG. 3 shows insertion of a head of a femoral component into the dual mobility bearing component of FIG. 1;

FIG. 4 shows a cross-sectional view of FIG. 3;

FIG. 5 shows articulation of the head of the femoral component within the dual mobility bearing component of FIG. 1;

FIG. 6 shows a cross-sectional view of FIG. 5;

FIG. 7 shows a plan view of the dual mobility bearing component;

FIG. 8 shows a side cross-sectional view of the dual mobility bearing component;

FIG. 9 shows a perspective cross-sectional view of the dual mobility bearing component;

FIG. 10 shows a magnified cross-sectional view of a rim of the dual mobility bearing component;

FIG. 11 shows a magnified view of the rim of the dual mobility bearing component of FIG. 2;

FIG. 12 shows a comparison of insertion and extraction forces of the present dual mobility bearing component as compared to conventional dual mobility bearing component; and

FIG. 13-18 show various embodiments of the present dual mobility bearing component wherein sub figures A thereof show a side cross-sectional view along a major axis, sub figures B thereof show a side cross-sectional view along a minor axis and sub figures C thereof show perspective views thereof.

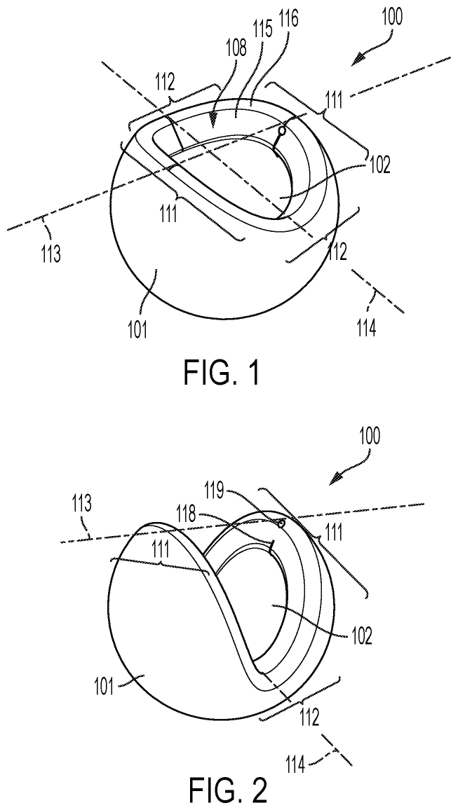

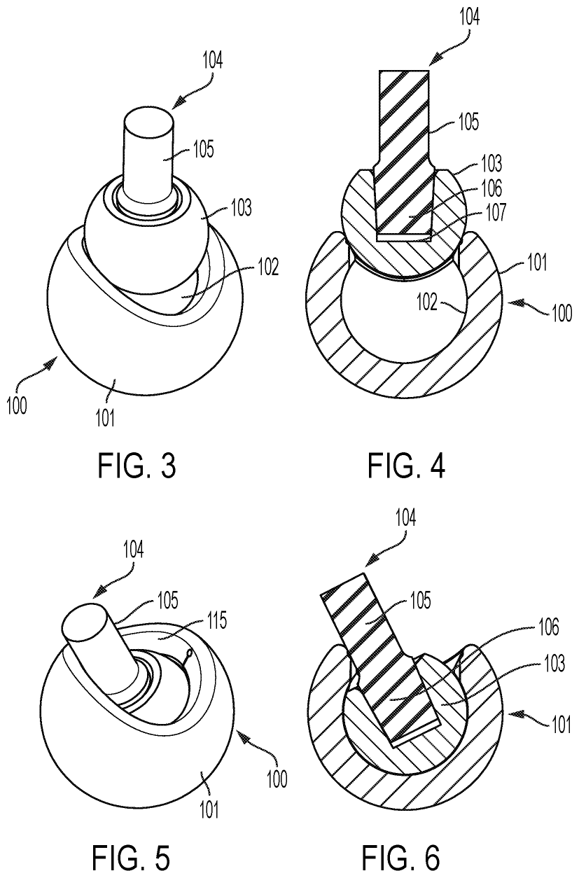

### DESCRIPTION OF EMBODIMENTS

There is provided herein hip prosthesis which comprises an acetabular component defining a semi-spherical inner bearing surface and a femoral component 104 having a head 103 defining a semi-spherical outer bearing surface.

The acetabular component may comprise an osteointegration metallic or other cup lined with a highly polished liner or surface.

The femoral component 104 may define a neck 105 having a proximal trunnion 106 configured for mortise interlock or similar within a blind aperture 107 defined by the head 103. The head 103 is typically metallic or ceramic and the neck 105 is typically metallic.

The prosthesis further comprises a dual mobility bearing component 100 which defines a semi-spherical outer bearing surface 101 freely articulating within the acetabular component thereby defining an outer bearing.

The dual mobility bearing component 100 further defines a semi-spherical inner bearing surface 102 wherein the spherical head 103 freely articulates within the inner bearing surface 102 thereby defining an inner bearing.

The bearing component 100 is typically polymeric, such as comprising High Density Polyethylene (HDPE).

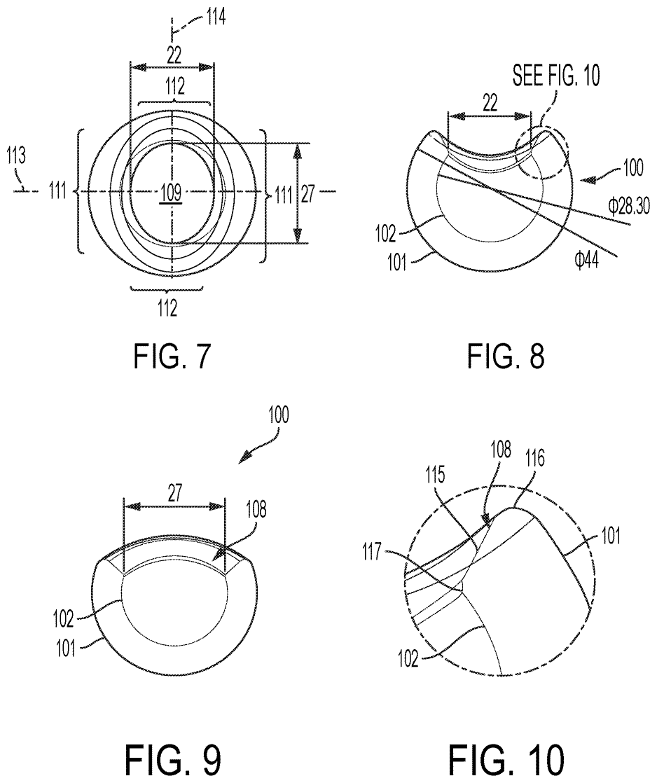

The bearing component 100 defines a rim 108 transitioning the bearing surfaces 101, 102 thereby defining an aperture 109 (best shown in FIG. 7) for the head 103 and neck 105 of the femoral component 104.

The rim 108 has a 3D geometry which can be generally described as high-sided and low-ended, much like if a sphere were transected by an ellipsoid. In other words, the rim 108 is higher at sides thereof as compared to at ends thereof.

More specifically, the rim 108 defines higher sides 111 across a minor axis 113 (e.g. relatively narrower axis of the aperture 109 according to the plan view shown in FIG. 7) and lower sides 112 across a major orthogonal axis 114 (e.g., relatively wider axis of the aperture 109). In other words, with reference to FIG. 7, the aperture 109 is wider along the major axis 114 as compared to along the minor axis 113.

“Freely articulating” refers to the ability of the bearing component 100 or the head 103 to move, rotate, or adjust with without restrictions (i.e. such as pins, coupling components or the like which would restrict movement) within the acetabular component or inner bearing surface 102 of the bearing component 100 respectively allowing them adapt to different positions or orientations. In other words, the bearing component 100 freely articulates within the acetabular component without mechanical restriction (such as pins, coupling components or the like) that would restrict movement of the bearing component 101 along a pivot axis with respect to the acetabular component.

The term “semi-spherical” refers to the bearing surface 101 or 102 being a partially spherical surface that has a curved or rounded form similar to that of a sphere but does not extend fully into a three-dimensional spherical shape, thereby allowing for free articulation.

As such, once implanted, the free articulation afforded by the semi-spherical bearing surfaces 101 and 102 allow the bearing component 100 to generally rotate and orientate within the acetabular component so that flexion and extension movement of the femoral component 104 predominates along the wider aspect of the major axis 114 whereas adduction and abduction movement of the femoral component 104 (induced to occur only at the outer bearing component 100 due to impingement of the neck 105 against the high sides of the rim 108 (i.e. along the minor axis 113) of the dual mobility bearing component 100) predominates along the relatively narrower aspect of the minor axis 113.

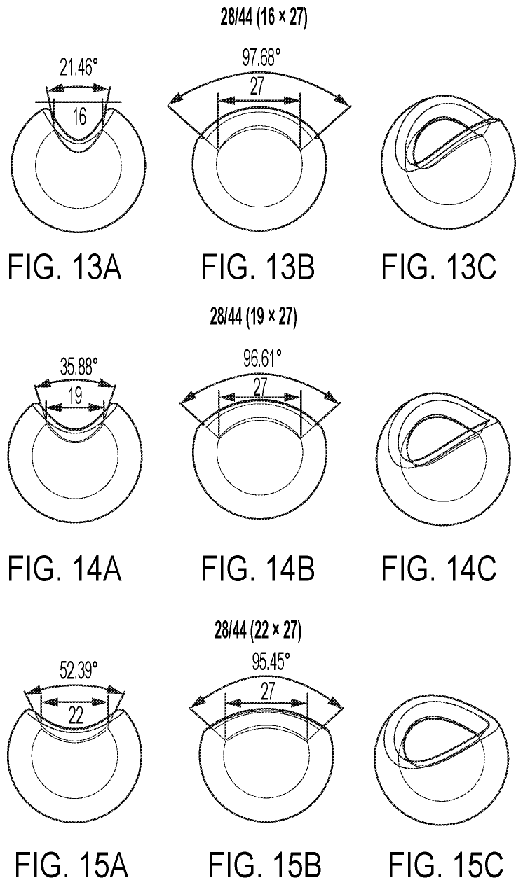

The angular extent of the lower ends 112 may be similar to that of conventional dual mobility bearing components which are designed to capture the femoral head 103 by more than 180° such as at about 240° to thereby capture the head 103 to prevent dislocation from the dual mobility bearing component, commonly referred to as known as dissociation. For example, FIG. 13B shows the angular extent of the lower ends 112 designed to capture the head 103 by approximately 260° and FIG. 17B shows the angular extent of the lower ends 112 designed to capture the head 103 by approximately 230°.

However, the higher sides 112 of the bearing component 100 are designed to capture the head 103 by much more than conventional dual mobility liners, such as by more than 270°, preferably more than 280° and, in embodiments, by more than 300°.

The effect of the capturing of the head 103 by the higher sides 111 is that the lower ends 112 may capture the head 103 through less than 180° in an embodiment as is shown in the embodiment of FIG. 18B.

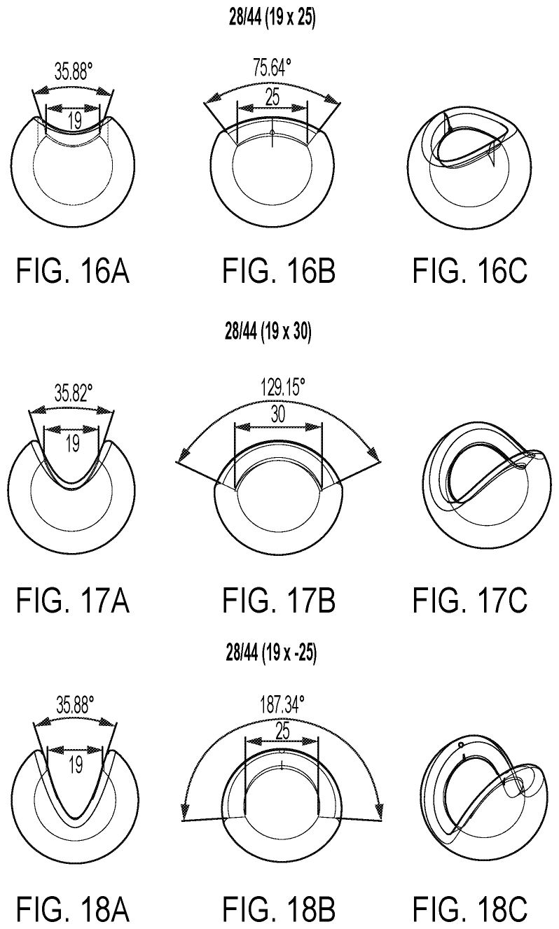

As is shown in FIGS. 13-18A , the rim 108 may define a parabolic cross-section along the major axis 114.

The embodiments of FIGS. 13-18 show a standard sized 28/44 bearing component 100, meaning that the bearing component 100 has an exterior diameter of approximately 44 mm and an interior diameter of approximately 28 mm. The associated dimensions shown in brackets with each figure represents the width of the aperture 109 along the minor axis 113 and the length of the aperture 109 along the major axis 114. For example, according to the embodiment of FIG. 13, the aperture 109 is 16 mm wide and 27 mm long.

In embodiments, the outer diameter of the bearing component 100 may be configured to fit and acetabular cup having an inner diameter of between 38-62 mm. Furthermore, the inner diameter of the bearing component 100 may have diameters including of 22 mm, 28 mm, 32 mm and 36 mm.

According to the embodiment shown, the aperture 109 may have a length of between 25-27 mm and a width of between approximately 16 mm-19 mm depending on the angular extents of the higher sides 111 and the lower sides 112.

The rim 108 may define an inner flat contact face 115 which is designed to flatly contact the neck 105 of the femoral component 104 to spread and minimise contact forces thereacross.

With reference to the various embodiments shown in FIGS. 13-18 , an arc angle defined by the inner flat contact face 115 may be between approximately 21° and 53° along the minor axis 113 and between approximately 97°-187° along the major axis 114.

With reference to FIG. 10, the rim 105 may define a rounded shoulder 116 interfacing the inner flat contact face 115 and the outer bearing surface 101. As is further shown in FIG. 10, the rim 108 may define a chamfered inner edge 117 of approximately 1-3 mm in width interfacing the inner flat contact face 115 and the inner bearing surface 102.

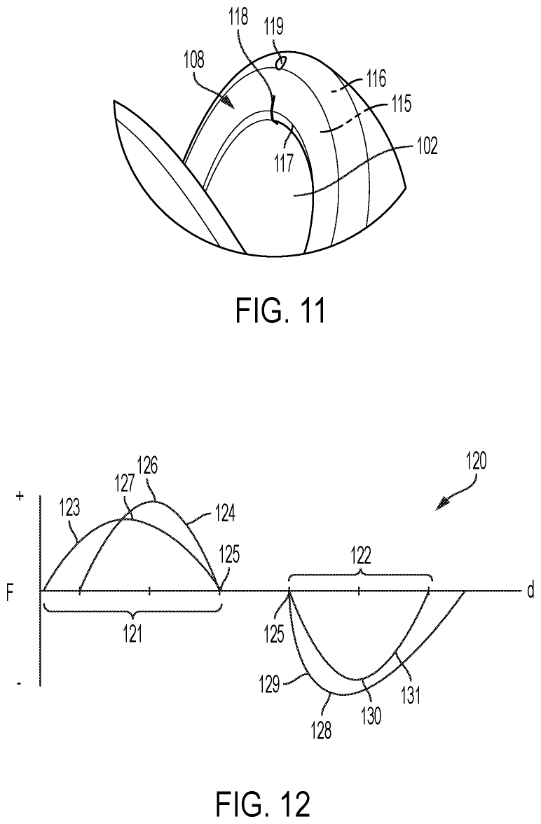

As shown in FIG. 11, the rim 108 may define expansion slits 118 transecting in the outer bearing surface 101 and inner flat contact face 115 and the inner bearing surface 102 along the minor axis 113. These expansion slits 118 allow the rim 108 to expand as the head 103 pushes past the chamfered edge 117 during insertion.

Each expansion slit 118 may terminate at one or more blind holes 119 which may arrest potential for polymeric crack propagation. The upper blind hole 119 shown in FIG. 11 may insert beyond the flat in a contact face 108, such as through the rounded shoulder 116 or the outer bearing surface 101. In embodiments, each expansion slit 118 may terminate in a lower blind hole at the inner bearing surface 102 In further embodiments, the rim 108 defines a locating dimple (not shown) for seating and locating the femoral head 103 prior insertion. The bearing component 100 may comprise a pair of location dimples approximately midway the flat contact face 115 along the minor axis 113.

FIG. 12 represents a comparison 120 of insertion and extraction forces of the present bearing component 100 as compared to a conventional liner having a coplanar rim.

The comparison 120 is shown with force along the vertical axis and with displacement along the horizontal axis.

The comparison 120 shows insertion forces 120 exerted to push the head 103 into the bearing component 100 until the head 103 is concentric with the inner bearing surface 103 at a concentric point 125 and extraction forces 122 to pull the head 103 from the bearing component 100.

When the head 103 is inserted into the present bearing component 100, sides of the head 103 contact the higher sides 113 before contacting the lower sides 112, thereby spreading insertion force 123 as compared to insertion force 124 of a conventional liner and exhibiting a lower maximum peak force 127 as compared to a maximum peak force 126 for a conventional liner.

During extraction 122, the greater envelopment of the head 106 by the higher sides 112 grips the head 103 more effectively than a conventional liner does, thereby exhibiting a steeper extraction force 129 having a greater maximum peak force 128 as compared to the maximum peak force 130 of extraction force 131 of a conventional liner. Furthermore, the greater envelopment of the head 106 by the higher sides 112 increases the necessary displacement for extraction or dissociation to occur.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. Hip prosthesis comprising:
an acetabular component defining a semi-spherical inner bearing surface;
a femoral component having a head defining a semi-spherical outer bearing surface;
a dual mobility bearing component, wherein the dual mobility bearing component defines:
a semi-spherical outer bearing surface freely articulating within the acetabular component thereby defining an outer bearing;
a semi-spherical inner bearing surface wherein the head freely articulates within the inner bearing surface thereby defining an inner bearing; and
a rim transitioning the bearing surfaces and defining an aperture for the head of the femoral component, wherein:
the rim is higher at sides thereof as compared to at ends thereof.

2. The prosthesis as claimed in claim 1, wherein, once implanted, the semi-spherical bearing surfaces allow the bearing component to orientate within the acetabular component so that flexion and extension movement of the femoral component predominates along a wider axis of the aperture and adduction and abduction movement of the femoral component predominates along a relatively narrower axis of the aperture.

3. The prosthesis as claimed in claim 2, wherein the adduction and abduction movement of the femoral component is induced to predominate at the outer bearing component due to impingement of a neck of the femoral component against high sides of the rim along the narrower axis of the aperture.

4. The prosthesis as claimed in claim 1, wherein higher sides of the rim are configured to capture the head by more than 270°.

5. The prosthesis as claimed in claim 1, wherein higher sides of the rim are configured to capture the head by more than 280°.

6. The prosthesis as claimed in claim 1, wherein higher sides of the rim are configured to capture the head by more than 300°.

7. The prosthesis as claimed in claim 1, wherein lower sides of the rim are configured to capture the head by less than 180°.

8. The prosthesis as claimed in claim 1, wherein higher sides of the rim cross a minor axis and lower sides of the rim cross a major axis and wherein the rim defines a parabolic cross section along the major axis.

9. The prosthesis as claimed in claim 1, wherein the aperture defines a cross-section having a length of between approximately 24 mm-28 mm and a width of between approximately 16 mm-22 mm.

10. The prosthesis as claimed in claim 1, wherein the rim defines an inner flat contact face configured for being parallel with an edge of a neck of a femoral component when contacting the neck.

11. The prosthesis as claimed in claim 10, wherein higher sides of the rim cross a minor axis and lower sides of the rim cross a major axis and wherein the inner flat contact face defines an arc angle of between approximately 21° and 53° across the minor axis by and an arc angle of between approximately 97°-187° along the major.

12. The prosthesis as claimed in claim 10, wherein the rim defines a rounded shoulder interfacing the inner flat contact face and the outer bearing surface.

13. The prosthesis as claimed in claim 10, wherein the rim defines a chamfered edge interfacing the inner flat contact face and the inner bearing surface.

14. The prosthesis as claimed in claim 1, wherein the rim has slits through the higher sides thereof.

15. The prosthesis as claimed in claim 1, wherein the rim defines an inner flat contact face and the rim has slits at higher sides thereof transecting the inner flat contact face and the inner bearing surface.

16. The prosthesis as claimed in claim 15, wherein each slit terminates at an blind hole.

17. The prosthesis as claimed in claim 15, wherein the upper blind hole is beyond the inner flat contact face.

18. The prosthesis as claimed in claim 1, wherein the rim defines an inner contact face and the rim has locating dimples in the inner contact face at higher sides thereof.

19. The prosthesis as claimed in claim 1, further comprising the femoral component and wherein the head thereof articulates against the inner bearing surface.

20. The prosthesis as claimed in claim 19, further comprising acetabular component and wherein the outer bearing surface articulates against the acetabular component.

21. The prosthesis as claimed in claim 20, wherein the acetabular component comprises an acetabular cup liner and wherein the outer bearing surface articulates against an inner bearing surface of the acetabular cup liner.

22. The prosthesis as claimed in claim 21, wherein the acetabular cup liner is constrained with a locking ring.

23. A method involving the prosthesis as claimed in claim 1, the method comprising implanting the acetabular component, dual mobility bearing component and femoral component so that the semi-spherical outer bearing surface of the dual mobility bearing component freely articulates within the acetabular component and the head freely articulates within the inner bearing surface of the dual mobility bearing component.

24. The method as claimed in claim 23, wherein the semi-spherical bearing surfaces allow the bearing component to orientate within the acetabular component so that flexion and extension movement of the femoral component predominates along a wider axis of the aperture and adduction and abduction movement of the femoral component predominates along a relatively narrower axis of the aperture.

25. The method as claimed in claim 23, wherein the adduction and abduction movement of the femoral component is induced to predominate at the outer bearing component due to impingement of a neck of the femoral component against high sides of the rim along the narrower axis of the aperture.
